(19) United States
(12) Patent Application Publication
(10) Pub. No.: US 2025/0195708 A1
(43) Pub. Date: Jun. 19, 2025

(54) STERILISING CONTAINER AND CONTROL DEVICE

(71) Applicant: Aesculap AG, Tuttlingen (DE)

(72) Inventors: Arne Jansen-Troy, Stockach (DE); Matthias Henke, Villingen-Schwenningen (DE); Daniel Kiessling, Villingen-Schwenningen (DE)

(21) Appl. No.: 18/847,372

(22) PCT Filed: Mar. 16, 2023
(86) PCT No.: PCT/EP2023/056836

§ 371 Date: Sep. 16, 2024

(30) Foreign Application Priority Data
Mar. 17, 2022 (DE) 10 2022 106 280.5

(51) Int. Cl. A61L 2/26; A61B 50/00; A61B 50/30; A61L 2/24

(52) U.S. Cl. CPC A61L 2/26; A61B 50/30; A61L 2/24; A61B 2050/0076; A61L 2202/121; A61L 2202/122; A61L 2202/123; A61L 2202/14; A61L 2202/24

(57) ABSTRACT

A sterilizing container for medical purposes includes a base and a lid fastenable to the base. When the lid is closed, the base and lid define an inner region of the sterilizing container. A media exchange mechanism is designed, when the lid is closed, to selectively open and close at least one fluid-mechanical connection between the inner region and a surrounding environment. A control device is designed to control the media exchange mechanism. The sterilizing container has a ventilation device designed to convey a medium in the inner region and/or in the surrounding environment of the sterilizing container to the media exchange mechanism. The control device is designed to control the media exchange mechanism and the ventilation device in such a way that the at least one fluid-mechanical connection is open when a medium is conveyed. A control device can externally control the control device of the sterilizing container.

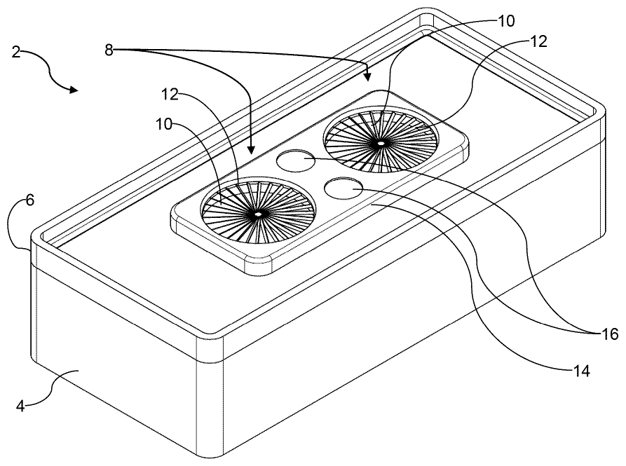

### CROSS-REFERENCE TO RELATED APPLICATIONS

This application is the United States national stage entry of International Application No. PCT/EP2023/056836, filed on Mar. 16, 2023, and claims priority to German Application No. 10 2022 106 280.5, filed on Mar. 17, 2022. The contents of International Application No. PCT/EP2023/056836 and German Application No. 10 2022 106 280.5 are incorporated by reference herein in their entireties.

### FIELD

The present disclosure relates to a sterilization container for medical purposes, in particular to a sterilization container whose lid is fixable to a bottom element so that the lid in its closed state together with the bottom element defines an inner region of the sterilization container from which at least one fluid-mechanical connection to surroundings of the sterilization container is formed which can be selectively opened and closed by a media exchange mechanism controlled by a control apparatus. The present disclosure further relates to a control device for externally controlling the control apparatus of the sterilization container.

### BACKGROUND

A generic sterilization container is known, for example, from WO 2020/074 657 A1 and has a bottom element and a lid. The lid can be fixed to the bottom element so that an inner region of the sterilization container is formed. Two openings are provided in the bottom element, each of which represents a fluid-mechanical connection from the inner region to the surroundings. The sterilization container according to WO 2020/074 657 A1 has valve units equipped with electromechanical actuators and via which the openings in the bottom element can be opened and closed in accordance with control commands from a control unit.

A disadvantage of the generic sterilization container is that instruments may still be very moist and/or very hot after completion of sterilization in the generic sterilization container, so that long post-drying phases may have to be included in an instrument cycle.

### SUMMARY

Against this background, the object of the present disclosure is to provide a sterilization container which requires no or only comparatively short post-drying phases.

This object is solved by a sterilization container and by a control device.

The sterilization container according to the disclosure is configured for medical purposes and is suitable in particular for holding medical or surgical instruments. The sterilization container has a bottom element, a lid, a media exchange mechanism and a control apparatus.

The bottom element is configured in particular to be trough-shaped.

The lid is configured to be attached to the bottom element in such a way that the lid and the bottom element define an inner region of the sterilization container (closed state of the lid).

The media exchange mechanism is configured to be able to selectively open and close at least one fluid-mechanical connection between the inner region and the surroundings during the closed state of the lid.

The media exchange mechanism may be configured exclusively on the lid, exclusively on the bottom element, or partially on the lid and partially on the bottom element. In particular, the fluid-mechanical connection is an opening. In order to be able to selectively open and close this opening, a valve body is provided in particular, which can preferably be moved via an electromechanical actuator. The control apparatus is configured, in particular, to be able to control the actuator in such a way that it can bring the valve body into a closed position, in which the valve body locks the at least one opening, in particular in a gas-tight and/or liquid-tight manner and/or to be impermeable to germs, and into an open position, in which the valve body enables a medium to flow from the inner region into surroundings and/or a medium to flow from the surroundings into the inner region. The valve body is configured as a filter that is air-permeable or gas-permeable but impermeable to germs. Preferably, the valve body or the filter is configured to be exchangeable or replaceable without tools.

According to the disclosure, the sterilization container comprises a ventilation apparatus configured to be able to convey a medium in the inner region via the media exchange mechanism into the surroundings of the sterilization container and/or a medium in the surroundings via the media exchange mechanism into the inner region of the sterilization container. In other words, the ventilation apparatus is configured to be able to convey a medium in the inner region and/or a medium in the surroundings of the sterilization container to the media exchange mechanism. The medium in the inner region and/or in the surroundings is in particular germ-free air. The air or the medium in the inner region is in particular moist. The air or the medium in the surroundings is in particular dry.

In particular, the ventilation apparatus has a fan that is arranged at the opening. The fan may preferably be configured as an axial fan or a radial fan.

The control apparatus of the sterilization container is configured, in particular, to be able to control the media exchange mechanism and the ventilation apparatus in such a way that the at least one fluid-mechanical connection is open when a medium is conveyed.

The control apparatus is configured according to the disclosure to be able to control the media exchange mechanism and the ventilation apparatus in such a way that the fluid- mechanical connection is open or opened when a medium in the inner region is conveyed into the surroundings, and the fluid-mechanical connection is closed when a medium in the surroundings is conveyed into the inner region via the filter that is impermeable to germs but air-permeable or gas-permeable.

In particular, the media exchange mechanism may be configured to be opened by the ventilation apparatus. In other words, the valve body or filter may be pre-stressed in its closed position such that it can be released by a negative pressure or pressure gradient generated by the ventilation apparatus.

The ventilation apparatus advantageously provides a means for actively exchanging a medium located in the inner region of the sterilization container. An active exchange may shorten or avoid a post-drying phase in an instrument circuit.

According to an aspect of the disclosure, the sterilization container may comprise a sensor apparatus. The sensor apparatus may be configured to be able to measure a pressure, a temperature and/or a humidity in the inner region of the sterilization container and/or in the surroundings of the sterilization container. The control apparatus may be configured to be able to control the media exchange mechanism and the ventilation apparatus taking into account one measuring value or more measuring values of the sensor apparatus. In particular, the control apparatus may be configured to be able to record measuring values of the sensor apparatus and to compare them with comparison values or comparison profiles stored in the control apparatus and to be able to actuate the media exchange mechanism and the ventilation apparatus at times that are predetermined on the basis of the comparison values or the comparison profiles.

If the sterilization container is equipped with a sensor apparatus, automatic control of a media exchange can be implemented in an advantageous way.

According to one aspect of the disclosure, the media exchange mechanism and the ventilation apparatus may be configured exclusively on the lid. In particular, the control apparatus and possibly also the sensor apparatus may also be configured exclusively on the lid.

If electronic or electromechanical components are configured exclusively on the lid, the bottom element can still be used in the event of a defect in these components.

According to an aspect of the disclosure, the media exchange mechanism may have two fluid-mechanical connections or openings, each of which is selectively openable and closeable, and the control apparatus may be configured to be able to control the ventilation apparatus such that the ventilation apparatus conveys a medium in the surroundings of the sterilization container to a first one of the two fluid-mechanical connections and conveys a medium in the inner region of the sterilization container to a second one of the two fluid-mechanical connections. In particular, a valve body or a filter is provided for each fluid-mechanical connection. In particular, the fluid-mechanical connections are arranged on two opposite sides of the sterilization container.

By providing two fluid-mechanical connections, an optimized media exchange can be realized in an advantageous way.

According to an aspect of the disclosure, the control apparatus may be configured to be able to control the ventilation apparatus such that the ventilation apparatus alternately conveys a medium in the surroundings of the sterilization container to the first fluid-mechanical connection and conveys a medium in the inner region of the sterilization container to the second fluid-mechanical connection on the one hand, and conveys a medium in the surroundings of the sterilization container to the second fluid-mechanical connection and conveys a medium in the inner region of the sterilization container to the first fluid-mechanical connection on the other hand.

If the sterilization container is configured in such a way that a flow direction can be varied, possibly existing dead zones can be minimized.

According to an aspect of the disclosure, the sterilization container may have a wirelessly rechargeable energy storage configured to be able to supply energy to the control apparatus, the media exchange mechanism and/or the ventilation apparatus. In particular, the energy storage may be configured as a battery or galvanic cell or accumulator. In particular, the energy storage is equipped with an induction coil, via which wireless charging of the energy storage is possible.

If the energy storage is configured in such a way that it can be charged wirelessly, it is advantageously possible to use an energy storage with a low energy storage capacity without having to compromise on functionality.

According to an aspect of the disclosure, the control apparatus may comprise a receiver configured to be able to receive actuation signals which cause the control apparatus to convey medium via the ventilation apparatus from the inner region and/or from the surroundings of the sterilization container. In particular, the receiver is configured to receive actuation signals wirelessly.

According to an aspect of the disclosure, the control apparatus may comprise a transmitter configured to be able to transmit status signals containing information about a status of the media exchange mechanism and/or of the ventilation apparatus. In particular, the transmitter is configured to be able to send status signals wirelessly.

Preferably, the transmitter and the receiver of the control apparatus are configured integrally in the form of a transceiver.

By providing a receiver and/or a transmitter, the degree of automation of a sterilization process can be increased and/or the monitoring of the sterilization process can be improved.

A control device according to the disclosure is suitable for externally controlling a control apparatus of a sterilization container according to the disclosure. ‘External’ means in particular that the control device is not part of the sterilization container or is not permanently connected to the sterilization container.

The control device according to the disclosure has a transmitter (transmitter of the control device in contrast to the transmitter of the control apparatus) which is configured to be able to transmit actuation signals to the control apparatus, and/or has a receiver (receiver of the control device in contrast to the receiver of the control apparatus) which is configured to be able to receive status signals from the control apparatus. Preferably, the transmitter and the receiver of the control device are configured integrally in the form of a transceiver. In particular, the control device is configured to be able to communicate wirelessly with the control apparatus of the sterilization container. In other words, the control device is configured in particular as a remote control for the sterilization container. In particular, the external control device is configured on or in an autoclave.

If an external control device is provided, the degree of automation of a sterilization process can be increased.

According to an aspect of the disclosure, the control device may comprise a charging apparatus configured to wirelessly charge an energy storage of a sterilization container according to the disclosure. In particular, the charging apparatus may comprise an induction coil that can interact with the induction coil of the control apparatus.

By providing a charging apparatus in the control device, the sterilization container can be used without separate charging times that take place outside of an instrument circuit.

### BRIEF DESCRIPTION OF THE DRAWING

The present invention is described in more detail below based on a preferred configuration example with reference to the accompanying FIGURE. The FIGURE shows a perspective view of a sterilization container according to the disclosure.

### DETAILED DESCRIPTION

The sterilization container 2 according to the disclosure shown in the FIGURE has a trough-shaped bottom element 4 and a lid 6. The sterilization container 2 has a substantially rectangular outline.

The FIGURE shows the sterilization container 2 in a closed state of the lid 6. In this state, the bottom element 4 and the lid 6 define an inner region (not shown). In the closed state, the lid 6 is connected to the bottom element 4 in a manner impermeable to germs via a seal (not shown) on the circumference of the lid 6 or on free rims of sidewalls of the bottom element 4.

Two openings 8 are provided in the lid 6, which can be opened and closed via a media exchange mechanism. The media exchange mechanism has two replaceable filters 10 that are impermeable to germs but permeable to air, which are arranged in the openings 8. In the state shown in the FIGURE, the filters 10 are in a closed position. In the closed position of the filters 10 and in the closed state of the lid 6, the inner region is locked impermeable to germs with respect to the surroundings of the sterilization container 2.

An axial fan 12 is provided above each of the two filters 10 or on an outer side of each of the two filters 10 facing the surroundings.

Each of the two axial fans 12 is enclosed by a control apparatus 14 or by a housing of the control apparatus 14. An energy storage unit (not shown) is provided in the control apparatus 14, which also supplies the electric motors (not shown) provided in the control apparatus with energy to drive the axial fans 12.

A sensor apparatus 16 with two sensors for measuring a temperature, a pressure and/or a humidity of the surroundings and the inner region is arranged on the outer side of the control apparatus 14 between the axial fans 12.

The control apparatus 14 is configured to control one of the two axial fans 12 in such a way that a pressure gradient is generated in the direction of the surroundings when a predetermined measuring value or several predetermined measuring values are measured by the sensor apparatus 16, and to control the other one of the two axial fans 12 in such a way that a pressure gradient is generated in the direction of the interior.

The filters 10 are configured in such a way that they open automatically when a pressure gradient in the direction of the surroundings is applied to the respective filter 10, and that they remain in the closed position when a pressure gradient in the direction of the inner region is applied to the respective filter 10. In this way, a medium or air that is conveyed into the inner region by an axial fan 12 is filtered by the corresponding filter 10. In contrast, a medium or air that is conveyed into the surroundings by an axial fan 12 is not filtered.

The control apparatus 14 is configured to control the one of the two axial fans 12 that conveys medium into the inner region in such a way that the flow resistance generated by the corresponding filter 10 is overcome. In particular, the axial fans 12 are controlled in such a way that a medium or air flow into the inner region is the same strength as a medium or air flow out of the inner region.

### LIST OF REFERENCE SIGNS

2 sterilization container
4 bottom element
6 lid
8 opening
10 filter
12 axial fan
14 control apparatus
16 sensor apparatus

1.-10. (canceled)

11. A sterilization container for medical purposes, the sterilization container comprising:
a bottom element;
a lid which is fixable to the bottom element and which, in a closed state of the lid, together with the bottom element defines an inner region of the sterilization container;
a media exchange mechanism configured to selectively open and close, in the closed state of the lid, at least one fluid-mechanical connection between the inner region and surroundings via a filter that is air-permeable or gas-permeable but impermeable to germs;
a control apparatus configured to control the media exchange mechanism; and
a ventilation apparatus configured to convey a first medium in the inner region and a second medium in the surroundings of the sterilization container to the media exchange mechanism,
the control apparatus being configured to control the media exchange mechanism and the ventilation apparatus in such a way that the at least one fluid-mechanical connection is open when the first medium in the inner region is conveyed into the surroundings and is closed when the second medium in the surroundings is conveyed into the inner region via the filter.

12. The sterilization container according to claim 11, further comprising a sensor apparatus configured to measure a value of one or more of pressure, temperature and humidity in the inner region and/or in the surroundings of the sterilization container, wherein the control apparatus is configured to control the media exchange mechanism and the ventilation apparatus, taking into account said value of one or more of pressure, temperature and humidity in the inner region and/or in the surroundings of the sterilization container.

13. The sterilization container according to claim 11, wherein the media exchange mechanism and the ventilation apparatus are configured on the lid.

14. The sterilization container according to claim 11, wherein:
the at least one fluid-mechanical connection comprises a first fluid-mechanical connection and a second fluid-mechanical connection, each of the first fluid-mechanical connection and the second fluid-mechanical connection being selectively openable and closable, and
the control apparatus is configured to control the ventilation apparatus such that the ventilation apparatus conveys the second medium in the surroundings of the sterilization container to the first fluid-mechanical connection and conveys the first medium in the inner region of the sterilization container to the second fluid-mechanical connection.

15. The sterilization container according to claim 14, wherein the control apparatus is configured to control the ventilation apparatus such that the ventilation apparatus alternatingly convey:
the second medium in the surroundings of the sterilization container to the first fluid-mechanical connection, and the first medium in the inner region of the sterilization container to the second fluid-mechanical connection, and
the second medium in the surroundings of the sterilization container to the second fluid-mechanical connection, and the first medium in the inner region of the sterilization container to the first fluid-mechanical connection.

16. The sterilization container according to claim 11, further comprising a wirelessly rechargeable energy storage configured to supply energy to the control apparatus and/or the media exchange mechanism and/or the ventilation apparatus.

17. The sterilization container according to claim 11, wherein the control apparatus comprises a receiver configured to receive actuation signals which cause the control apparatus to convey media via the ventilation apparatus from the inner region and/or from the surroundings of the sterilization container.

18. A control device for externally controlling the control apparatus of the sterilization container according to claim 17, the control device comprising at least one of:
a control transmitter configured to transmit actuation signals to the control apparatus; and
a control receiver configured to receive status signals from the control apparatus.

19. The sterilization container according to claim 11, wherein the control apparatus comprises a transmitter configured to transmit status signals containing information about a status of the media exchange mechanism and/or of the ventilation apparatus.

20. A control device for externally controlling the control apparatus of the sterilization container according to claim 19, the control device comprising at least one of:
a control transmitter configured to transmit actuation signals to the control apparatus; and
a control receiver configured to receive status signals from the control apparatus.

21. The control device according to claim 20, wherein:
the sterilization container comprises a wirelessly rechargeable energy storage configured to supply energy to the control apparatus and/or the media exchange mechanism and/or the ventilation apparatus, and
the control device comprises a charging apparatus for wirelessly charging the wirelessly rechargeable energy storage.
